(19) United States
(12) Patent Application Publication
(10) Pub. No.: US 2021/0077294 A1
(43) Pub. Date: Mar. 18, 2021

(54) STRETCHABLE HEATING TAPE

(71) Applicant: Sunbeam Products, Inc., Boca Raton, FL (US)

(72) Inventors: Lalit Mandal, Boca Raton, FL (US); Jill Walters, Delray Beach, FL (US)

(21) Appl. No.: 16/961,287

(22) PCT Filed: Jan. 11, 2019
(86) PCT No.: PCT/US2019/013327

§ 371 Date: Jul. 10, 2020

Related U.S. Application Data
(60) Provisional application No. 62/616,575, filed on Jan. 12, 2018.

(51) Int. Cl. A61F 7/00; A61F 7/02

(52) U.S. Cl. CPC A61F 7/007; A61F 7/02; A61F 2007/0078; A61F 2007/0226; A61F 2007/0285; A61F 2007/0071

(57) ABSTRACT

The present disclosure is directed to a heating mechanism including stretchable heating tape and a rechargeable electrical power source. The stretchable heating tape can be worn to provide heat and pressure relief on different parts of a user's body anisotropically. On one side of such stretchable tape lies a single-layer fabric in which non-stretchable heating wire is at least partially weaved with knitted stretchable yarns to make a stretchable fabric that can stretch up to 100%. On the other side of the stretchable tape lies an adhesive material that provides tension at various levels of stretch up to 100% and can sustain multiple hours on direct skin applications. Such stretchable fabric can have a connector coming from one end or both ends which provides a path for power through an inter-connecting cable between the stretchable tape and a controller which includes the power source and its control circuitry.

### CROSS-REFERENCE TO RELATED APPLICATIONS

This application claims the benefit of U.S. Provisional Application No. 62/616,575, entitled ELECTRICALLY HEATED TAPE, filed Jan. 12, 2018, which is hereby incorporated by reference in its entirety.

### FIELD

This invention relates generally to stretchable tape, such as kinesiology tape, and in particular, to stretchable tape having an electrical heating element.

### BACKGROUND

Kinesiology tape consists of a strip of elastic and non-elastic fibers, usually covered in cotton, which is placed on human skin. Kinesiology tape is useful in therapy to reduce soreness in overused and injured muscles and in rehabilitation to accelerate recovery. The tape can have a lifting effect on the skin which can reduce swelling and inflammation by improving circulation and reduce pain by taking pressure off pain receptors.

### SUMMARY

The present disclosure is directed to a heating mechanism including stretchable heating tape and a rechargeable electrical power source. The stretchable heating tape can be worn to provide heat and pressure relief on different parts of a user's body anisotropically. According to a first embodiment, a stretchable heating tape includes an elastic backing material, a continuous heating element at least partially woven through the elastic backing material, an adhesive material, and two or more heating element connection points. The heating element connection points are positioned on a first side of the elastic backing material and the adhesive material is positioned on a second side of the elastic backing material. The heating element connection points enable connection of an electric controller to the continuous heating element such that electric current from the electric controller is at least partially converted to heat energy when flowing through the continuous heating element. In an aspect, the elastic backing material and/or the adhesive material are configured to at least partially transfer the heat energy to a portion of a user's body when the stretchable heating tape is positioned adjacent to the portion of the user's body.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 depicts a top view of the stretchable heating tape;

FIG. 2A depicts another top view of the stretchable heating tape;

FIG. 2B depicts a bottom view of the stretchable heating tape;

FIG. 3 depicts a cross-sectional view of the stretchable heating tape of FIGS. 1, 2A, and 2B;

FIG. 4A depicts a top view of another embodiment of the stretchable heating tape;

FIG. 4B depicts a bottom view of the embodiment of the stretchable heating tape of FIG. 5A;

FIG. 5A depicts a top view of yet another embodiment of the stretchable heating tape;

FIG. 5B depicts a bottom view of the embodiment of the stretchable heating tape of FIG. 6A;

FIG. 6A depicts a bottom view of the stretchable heating tape of FIG. 1 in an unstretched state;

FIG. 6B depicts the bottom view of the stretchable heating tape of FIG. 1 in a stretched state;

FIG. 6C depicts a close-up of the bottom view of the stretchable heating tape of FIG. 1 in the unstretched state;

FIG. 6D depicts a close-up of the bottom view of the stretchable heating tape of FIG. 1 in the stretched state;

FIGS. 7 and 8 depict knitting diagrams for knitting an elastic backing material and weaving therein a conductive heating element of the stretchable heating tape;

FIG. 9 depicts a block diagram schematic of a controller for use with the stretchable heating tape;

FIG. 10 is an exploded perspective view of the controller and a controller housing;

FIG. 11 is a front perspective view of the controller housing;

FIG. 12 is a front perspective view of the controller housing and at least one electrical connector;

FIG. 13 is a rear perspective view of the controller housing;

FIG. 14A depicts a bottom view of another embodiment of the stretchable heating tape in an unstretched state;

FIG. 14B depicts a bottom view of the embodiment of the stretchable heating tape of FIG. 14A in a stretched state.

While the disclosure is susceptible to various modifications and alternative forms, specific embodiments thereof are shown by way of example in the drawings and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure.

### DETAILED DESCRIPTION

The present disclosure is directed to an on-the-go heating mechanism for effective heat with stretchable heating tape that can be worn to provide heat and pressure relief on different parts of a user's body anisotropically. As used herein, kinesiology tape refers to a fabric tape, having an adhesive on one side, with anisotropic stretching properties such that the kinesiology tape can be stretched in a first direction without significantly affecting the dimensions of the kinesiology tape in other unstretched dimensions. The stretchable heating tape described herein can use a rechargeable electrical power source (e.g., battery, USB power source, etc.) to provide electric current to a heating wire at least partially woven into and through what in some embodiments may be a single-layer stretchable tape. On one side of such stretchable tape lies a single-layer fabric in which non-stretchable heating wire is at least partially weaved with stretchable knitted yarns to make a stretchable fabric that can stretch up to 100%. On the other side of the stretchable tape lies an adhesive material that provides tension at various levels of stretch up to 100% and can sustain multiple hours on direct skin applications (e.g., up to 10 hours). Such stretchable fabric can have a connector coming from one end or both ends which will provide a path for electrical power through one or more inter-connecting cables between the stretchable heating tape and the controller which houses the electrical power source and its control circuitry.

A design feature of the present disclosure is a single-layer fabric in which non-stretchable heating wire is at least partially woven in a serpentine pattern with stretchable knitted yarn to make a stretchable fabric in horizontal, vertical, or both directions and stretches up to 100%. In knitting such fabric, portions of the heating wire can be placed at a fixed distance apart in-between stretchable yarns. In addition, the serpentine pattern of the heating wire can be placed horizontally, vertically, or any fixed angle relative to the single-layer fabric. The end points of such stretchable fabric can come from both sides or from a single side.

The present disclosure will now be described with reference to the drawing figures, in which like reference numerals refer to like parts throughout. For purposes of clarity in illustrating the characteristics of the present disclosure, proportional relationships of the elements have not necessarily been maintained in the drawing figures.

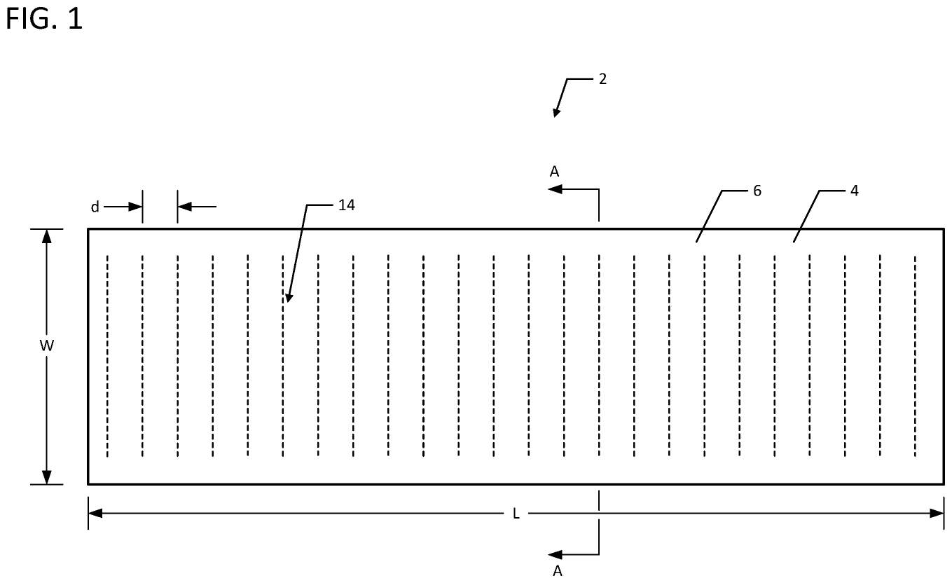

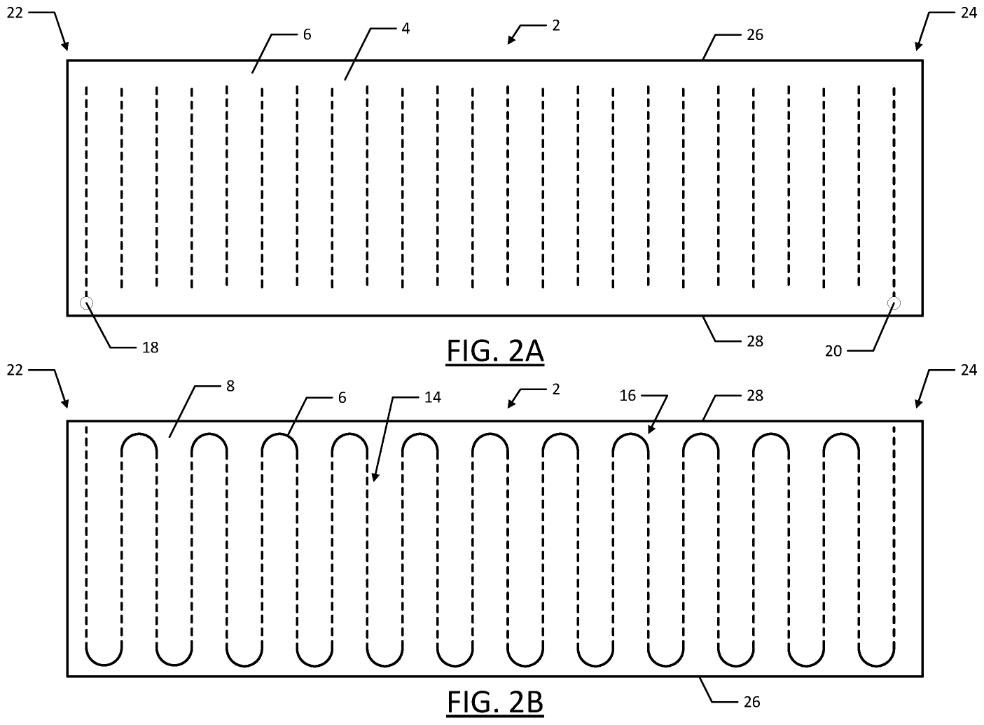

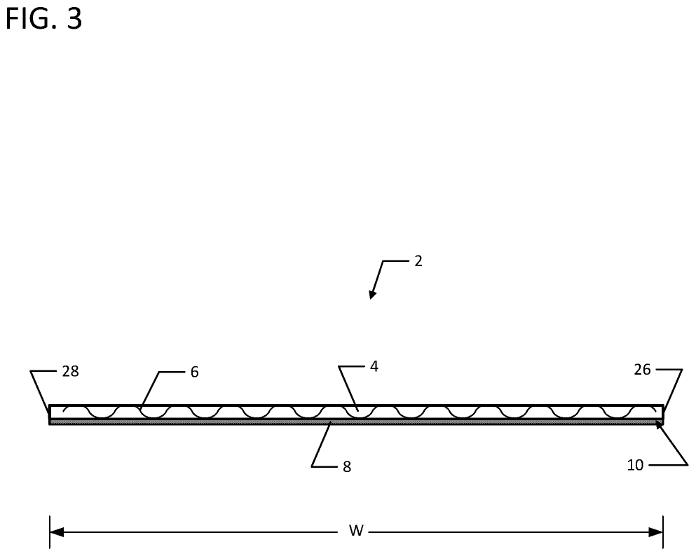

The present disclosure is directed to a stretchable heating tape 2. Referring now to the drawing figures in which like reference designators refer to like elements, there is shown in FIGS. 1-3 views of a stretchable heating tape 2 in accordance with the present disclosure. FIG. 1 is a top plan view of the stretchable heating tape 2. FIG. 2A is another top plan view of the stretchable heating tape 2 and FIG. 2B is a bottom plan view of the stretchable heating tape 2. FIG. 3 is a cross-sectional view of the stretchable heating tape 2 taken along the line A-A indicated in FIG. 1. The stretchable heating tape 2 includes an elastic backing material 4 having a conductive heating element 6 at least partially woven through the length “L” of the elastic backing material 4. An adhesive material 8 is provided on one side 10 of the stretchable heating tape 2 to allow for a placement/securement to a portion of a user's body. In an embodiment, the stretchable heating tape 2 has a thickness of three millimeters or less to enable the tape to fit discreetly under a user's clothing. An electric controller 12 is selectively connectable to the conductive heating element 6, such that when an electric current is supplied from the electric controller 12 through the conductive heating element 6, heat is produced. The produced heat is transferable from the conductive heating element 6 through the elastic backing material 4 and/or the adhesive material 8 to the portion of the user's body upon which the stretchable heating tape 2 is placed/secured.

The elastic backing material 4 can be made of a natural or synthetic knitted fabric that is stretchable along its length “L” and width “W.” In an embodiment, elastic backing material 4 is comprised of a nylon/spandex fabric (e.g., 17% spandex, 83% nylon). In some embodiments, the elastic backing material 4 is comprised of a plurality of interlocking loops made from one or more yarns, with each row of loops caught into the preceding row. A plurality of the interlocked loops may run in wales in the direction of the length “L” of the elastic backing material 4 and a plurality of the interlocked loops may run in courses in the direction of the width “W” of the elastic backing material 4. In some embodiments, the elastic backing material 4 is knitted using a jersey (i.e., plain) stitch. In further embodiments, the jersey stitch may be a birds eye jersey stitch. Additional details regarding the construction and manufacture of elastic backing material 4 are further described herein.

The conductive heating element 6 is at least partially woven through the elastic backing material 4 along the length “L” of the elastic backing material 4. For example, the conductive heating element 6 may be woven through interlocking loops of elastic backing material 4 that comprise a course (i.e., a row) in the direction of the width “W” of the stretchable heating tape 2. In some embodiments, conductive heating element 6 may be referred to as being integrated into or embedded within elastic backing material 4. In an embodiment, the conductive heating element 6 is woven in a serpentine pattern having a substantially vertical orientation with respect to the length “L” of the elastic backing material 4, such that a plurality of woven segments 14 of the conductive heating element 6 each span a width “W” of the elastic backing material 4 and are spaced apart a distance “d” along the length “L” of the elastic backing material 4. The spaced apart distance “d” can be such that adjacent woven segments 14 are in close proximity without abutting each other. In an exemplary embodiment, adjacent woven segments 14 are spaced from a distance “d” of 0.5 inches from each other up to a distance “d” of 1.5 inches from each other. However, it will be appreciated by persons skilled in the art that adjacent woven segments 14 can be spaced any distance “d” from each other that provides uniform heat distribution through stretchable heating tape 2. Each of the adjacent woven segments 14 are connected with an unwoven section 16, visible from the bottom of the elastic backing material 4, forming the continuous conductive heating element 6 in the serpentine pattern through the length “L” of the elastic backing material 4. In some embodiments, the unwoven sections 16 are arcuate. Referring particularly to FIG. 2B, the adjacent woven segments 14 of the conductive heating element 6 are woven into the elastic backing material 4, as illustrated by the dashed line portions, and the unwoven sections 16 of the conductive heating element 6 are not woven into the elastic backing material 4, as illustrated by the solid line portions. In this manner, the conductive heating element 6 is at least partially woven into the elastic backing material 4. For example, the conductive heating element 6 may be at least partially woven in a serpentine pattern. Moreover, each unwoven section 16 is unconstrained by the elastic backing material 4, which enables the conductive heating element 6 to remain continuous (i.e., not break) as the stretchable heating tape 2 is stretched. In embodiments in which the unwoven sections 16 are arcuate, the degree of curvature of each unwoven section 16 changes (e.g., flattens) as the stretchable heating tape 2 is stretched in the direction of its length “L”, as further described herein. In some embodiments, conductive heating element 6 may be referred to as a conductive heating thread.

Referring again to FIGS. 1-3 generally, the conductive heating element 6 is electrically coupled to heating element connectors 18 and 20 for connecting the conductive heating element 6 to an electrical connector that in turn is connected to the electric controller 12. In an embodiment, opposite free ends of the conductive heating element 6 are respectively coupled to the heating element connectors 18 and 20, which are positioned on opposite ends 22 and 24, respectively, of the elastic backing material 4. However, it is envisioned that the heating element connectors 18 and 20 can be positioned at the same end of the electric backing material 4, or alternatively on the same or opposite side edges 26 and 28 of the elastic backing material 4. In an embodiment, heating element connectors 18 and 20 each are comprised of a direct current (DC) jack female connector. However, it will be appreciated by persons skilled in the art that heating element connectors 18 and 20 may be any connectors capable of electrically coupling the conductive heating element 6 to an electrical connector that in turn is connected to the electric controller 12.

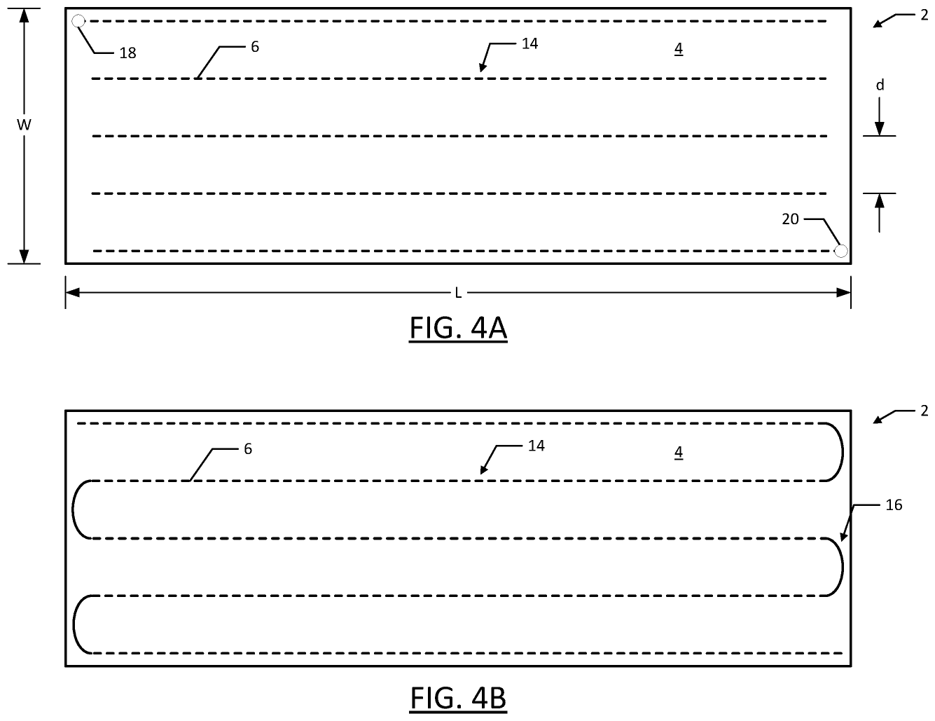

In another embodiment, illustrated in FIGS. 4A and 4B, the conductive heating element 6 is woven in a serpentine pattern having a substantially horizontal orientation along the width “W” of the elastic backing material 4, such that the plurality of woven segments 14 of the conductive heating element 6 each span at least a portion the length “L” of the elastic backing material 4 and are spaced apart a distance “d.” For example, the conductive heating element 6 may be woven through interlocking loops of elastic backing material 4 that comprise a wale (i.e., a column) in the direction of the length “L” of the stretchable heating tape 2. The spaced apart distance “d” can be such that adjacent woven segments 14 are in close proximity without abutting each other. In an exemplary embodiment, adjacent woven segments 14 are spaced from a distance “d” of 0.5 inches from each other up to a distance “d” of 1.5 inches from each other. However, it will be appreciated by persons skilled in the art that adjacent woven segments 14 can be spaced any distance “d” from each other that provides uniform heat distribution through stretchable heating tape 2. Each of the adjacent woven segments 14 are connected with an unwoven section 16, visible from the bottom of the elastic backing material 4, forming the continuous conductive heating element 6 in the serpentine pattern through the length “L” of the elastic backing material 4. In some embodiments, the unwoven sections 16 are arcuate. The adjacent woven segments 14 of the conductive heating element 6 are woven into the elastic backing material 4, as illustrated by the dashed line portions, and the unwoven sections 16 of the conductive heating element 6 are not woven into the elastic backing material 4, as illustrated by the solid line portions. In this manner, the conductive heating element 6 is at least partially woven into the elastic backing material 4. For example, the conductive heating element 6 may be at least partially woven in a serpentine pattern. Moreover, each unwoven section 16 is unconstrained by the elastic backing material 4, which enables the conductive heating element 6 to remain continuous (i.e., not break) as the stretchable heating tape 2 is stretched. In embodiments in which the unwoven sections 16 are arcuate, the degree of curvature of each unwoven section 16 changes (e.g., flattens) as the stretchable heating tape 2 is stretched in the direction of its width “W”, as further described herein.

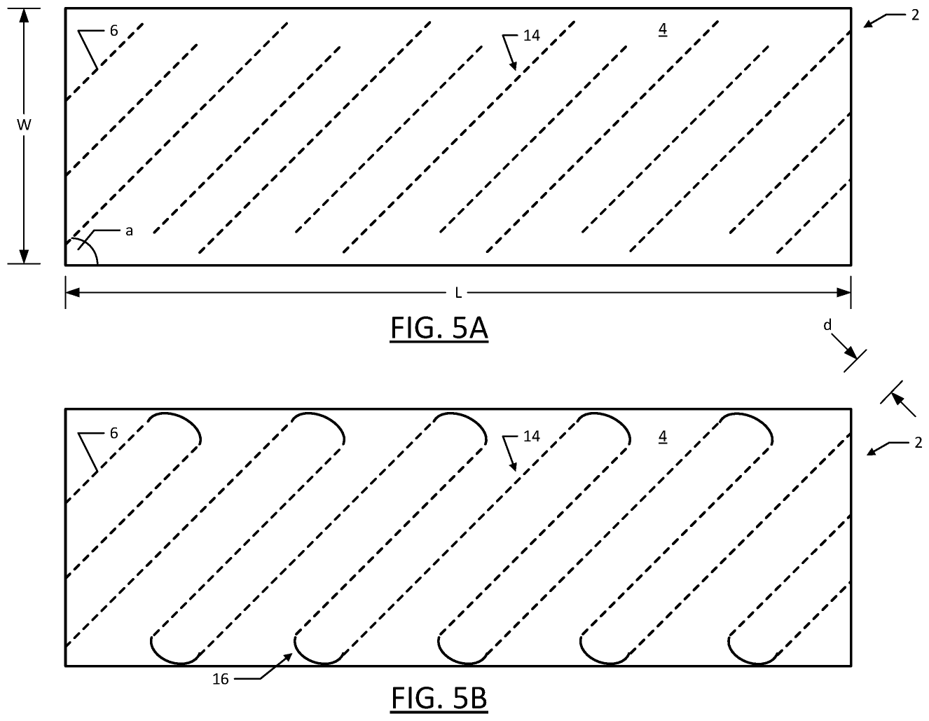

In a further embodiment, illustrated in FIGS. 5A and 5B, the conductive heating element 6 is woven in a serpentine pattern at an angle “a” with respect to the length “L” of the elastic backing material 4, such that a plurality of woven segments 14 of the conductive heating element 6 each are oriented along the length “L” and width “W” of the elastic backing material 4 and are spaced apart a distance “d.” The angle “a” can be any angle between one degree and eighty-nine degrees. In some embodiments, the angle “a” is thirty degrees. In other embodiments, the angle “a” is forty-five degrees. In further embodiments, the angle “a” is sixty degrees. The spaced apart distance “d” can be such that adjacent woven segments 14 are in close proximity without abutting each other. In an exemplary embodiment, adjacent woven segments 14 are spaced from a distance “d” of 0.5 inches from each other up to a distance “d” of 1.5 inches from each other. However, it will be appreciated by persons skilled in the art that adjacent woven segments 14 can be spaced any distance “d” from each other that provides uniform heat distribution through stretchable heating tape 2. Each of the adjacent woven segments 14 are connected with an unwoven section 16, forming the continuous conductive heating element 6 in the serpentine pattern through the length of the elastic backing material 4. In some embodiments, the unwoven sections 16 are arcuate. The adjacent woven segments 14 of the conductive heating element 6 are woven into the elastic backing material 4, as illustrated by the dashed line portions, and the unwoven sections 16 of the conductive heating element 6 are not woven into the elastic backing material 4, as illustrated by the solid line portions. In this manner, the conductive heating element 6 is at least partially woven into the elastic backing material 4. For example, the conductive heating element 6 may be at least partially woven in a serpentine pattern. Moreover, each unwoven section 16 is unconstrained by the elastic backing material 4, which enables the conductive heating element 6 to remain continuous (i.e., not break) as the stretchable heating tape 2 is stretched. In embodiments in which the unwoven sections 16 are arcuate, the degree of curvature of each unwoven section 16 changes (e.g., flattens) as the stretchable heating tape 2 is stretched in the direction of its length “L” and/or its width “W”, as further described herein.

Referring again to FIGS. 1-3, the conductive heating element 6 can be in the form of a conductive wire or conductive thread. For example, the conductive heating element 6 may be comprised of a thin copper wire. However, it will be appreciated by persons skilled in the art that the conductive heating element 6 can be comprised of any material that conducts electric current but also has electrical resistivity such that the electric current is at least partially converted to heat energy when flowing through the conductive heating element 6. In an embodiment, the conductive heating element 6 is a non-stretchable conductive wire or conductive thread that is woven with stretchable yarns through a single layer of the elastic backing material 4. In some embodiments, the conductive heating element 6 is electrically insulated with an insulating material (e.g., a sheath, jacket, etc.) that prevents electric current from flowing from the conductive heating element 6 to other components of the stretchable heating tape 2 and/or the user's body but also conducts heat energy generated by the conductive heating element 6 to other components of the stretchable heating tape 2 and/or portions of the user's body.

The adhesive material 8 is provided on one side 10 of the elastic backing material 4 to allow for a placement/securement of the stretchable heating tape 2 to a portion of a user's body. The adhesive material 8 is elastic in nature such that the adhesive material can be stretched simultaneously with the elastic backing material 4 without separation therefrom. The adhesive material 8 is also thermally conductive, allowing for the transfer of heat from the conductive heating element 6 to a portion of the user's body. In an embodiment, the adhesive material 8 is a silicone gel. For example, the adhesive material 8 may be the P-DERM® PS-2051 silicone gel/acrylic trilaminate available from Polymer Science, Inc. However, it will be appreciated by persons skilled in the art that the adhesive material 8 may be any adhesive material that can be stretched simultaneously with the elastic backing material 4 without separation therefrom, is thermally conductive, and allows for placement/securement of the stretchable heating tape 2 to a portion of a user's body.

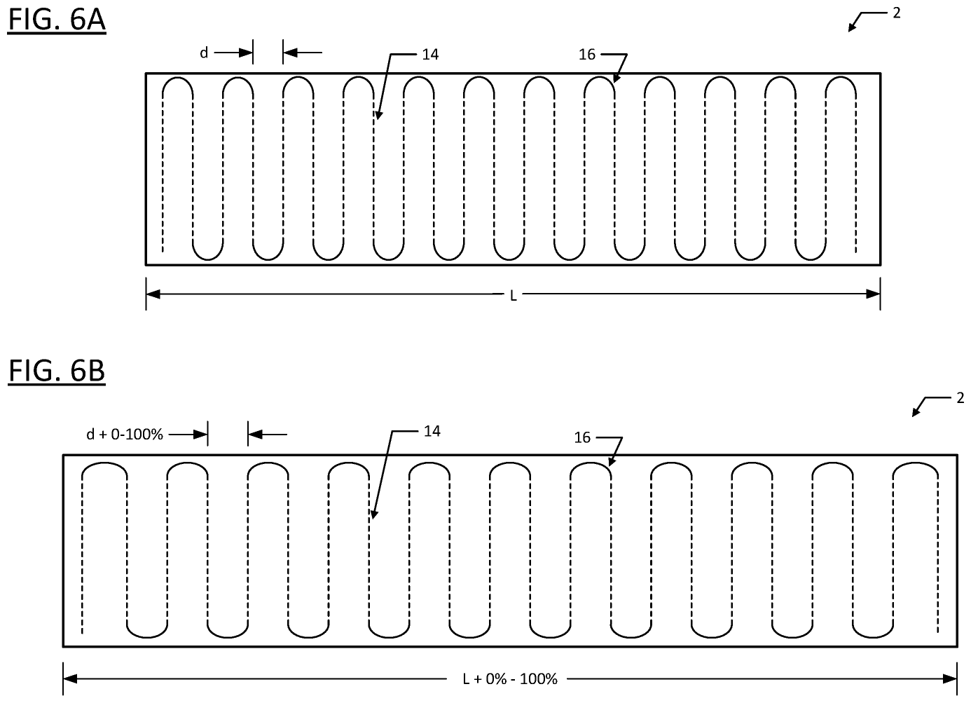

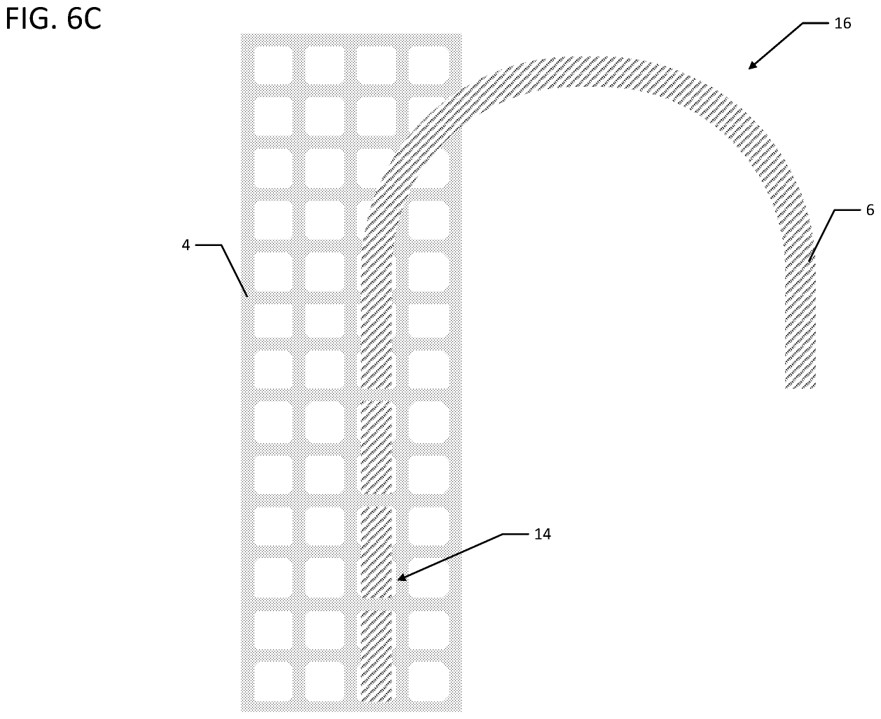

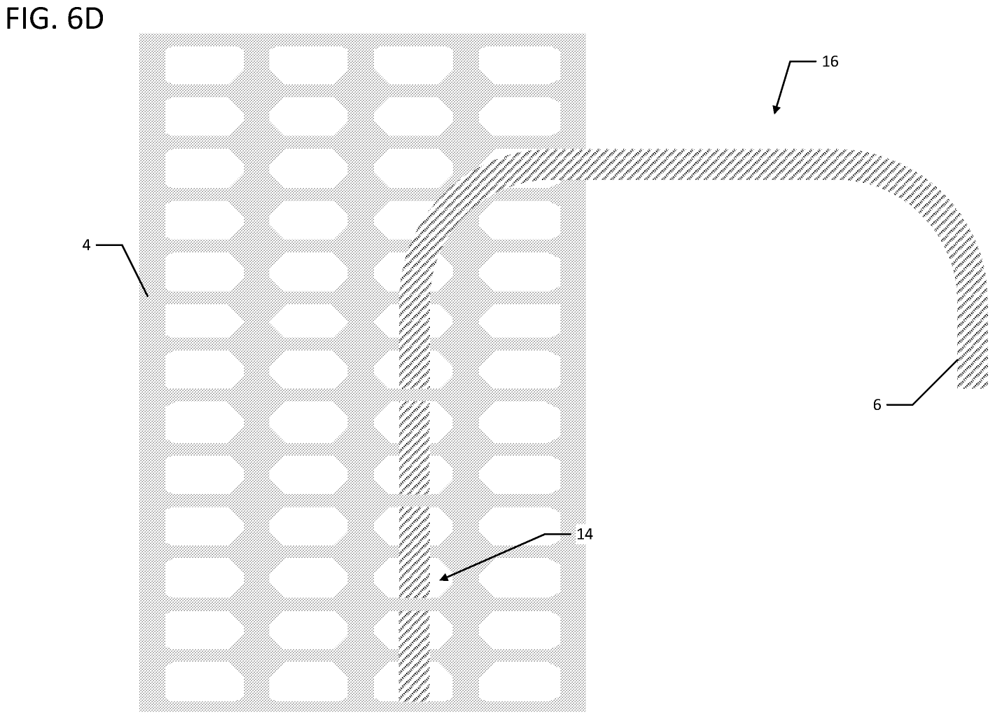

As illustrated in FIGS. 6A and 6B, the stretchable heating tape 2 may be stretchable from 0%-100% of its non-stretched length “L”. In an embodiment, the stretchable heating tape 2 has 0.125 pounds of pull force when stretched 20% of its non-stretched length, 0.875 pounds of pull force when stretched 40% of its non-stretched length, 1.125 pounds of pull force when stretched 60% of its non-stretched length, and 2.25 pounds of pull force when stretched 80% of its non-stretched length. Because the unwoven sections 16 of the conductive heating element 6 are not woven into the elastic backing material 4, the unwoven sections 16 are unconstrained and able to accommodate stretching of the stretchable heating tape 2 without breaking. In the embodiment illustrated in FIGS. 6A and 6B, the unwoven sections 16 are arcuate and the degree of curvature is able to change (e.g., flatten) as the stretchable heating tape 2 is stretched and the distance “d” between adjacent woven segments increases. The flattening of the unwoven sections 16 during stretching of the stretchable heating tape 2 allows the adjacent woven segments 14 to remain parallel to each other. Furthermore, this freedom in the degree of curvature of the unwoven sections 16 allows the conductive heating element 6 to maintain continuity (i.e., not break) as the stretchable heating tape 2 is stretched. FIGS. 6C and 6D illustrate a close-up view of an example of the change in the degree of curvature in an arcuate unwoven section 16 as the stretchable heating tape 2 is stretched.

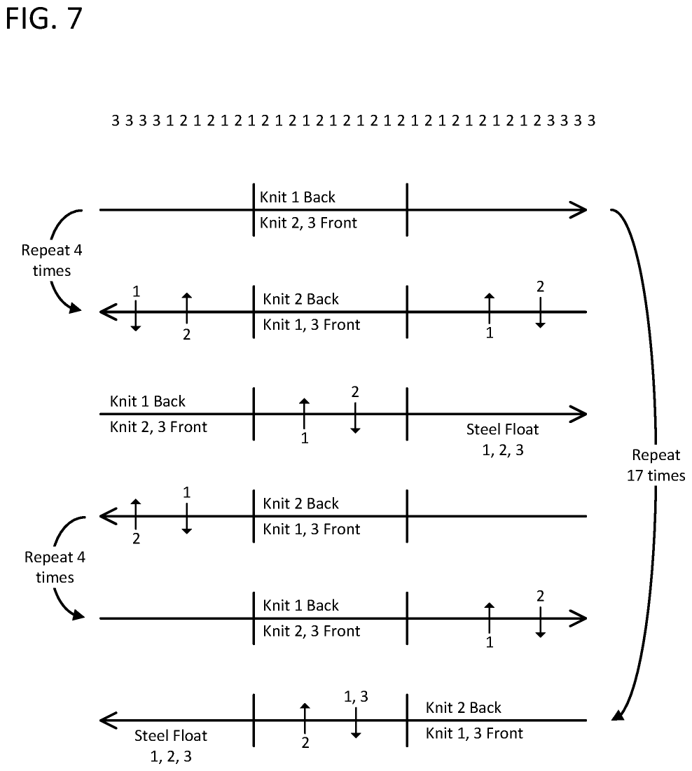

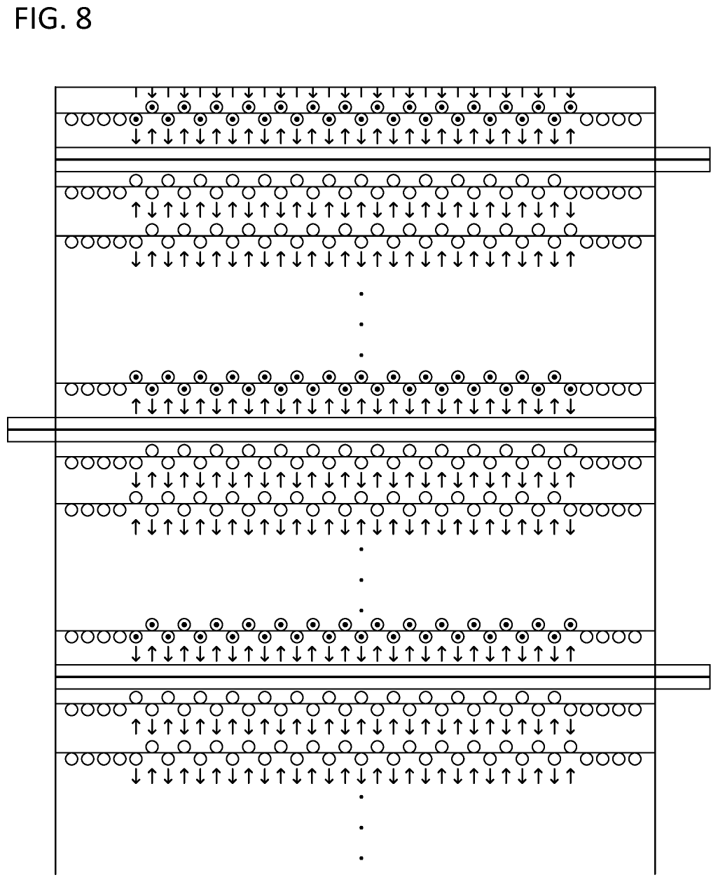

FIGS. 7 and 8 each illustrate an exemplary embodiment of a knitting diagram for manufacturing elastic backing material 4 and inlaying the conductive heating element 6. Table 1 describes properties for this exemplary embodiment, which results in one knit piece of nylon/spandex fabric (17% spandex, 83% nylon) (e.g., elastic backing material 4) measuring two inches by ten inches with an inlayed insulated wire (e.g., conductive heating element 6). In this embodiment, the knitting machine is preferably run with low and consistent take down and a bind off technique used to finish the knit product on a raw edge.

TABLE 1

| | |
|---|---|
| Knitting Machine | CMS ADF32 W E7.2 knitting machine |
| Type | available from H. Stoll AG & Co. KG |
| Knit Gauge | 14 GG |
| Yarn Type | 140 DC 1/70/34 Nylon 6.6 |
| Yarn Description | 140 Denier Spandex Core conventionally |
| | covered with two ends of 1/70/34 “Reflexx” |
| | nylon available from Unifi, Inc. |
| Yarn Usage | 4 grams |
| Wire Type | Insulated copper wire |
| Wire Usage | 68″ (5′ 8″ LF) |
| Knitting Structure | Birds Eye Jersey |
| Accessories | Elastic Feeder to control feed of |
| | 140 DC 1/70/34 yarn; Tension |
| | setting of 5 grams |

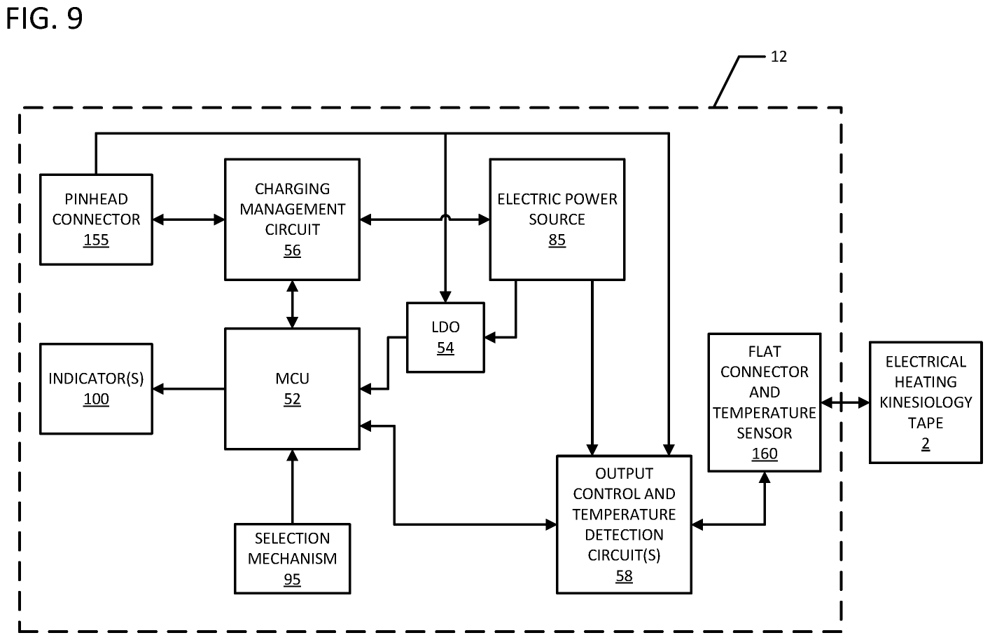

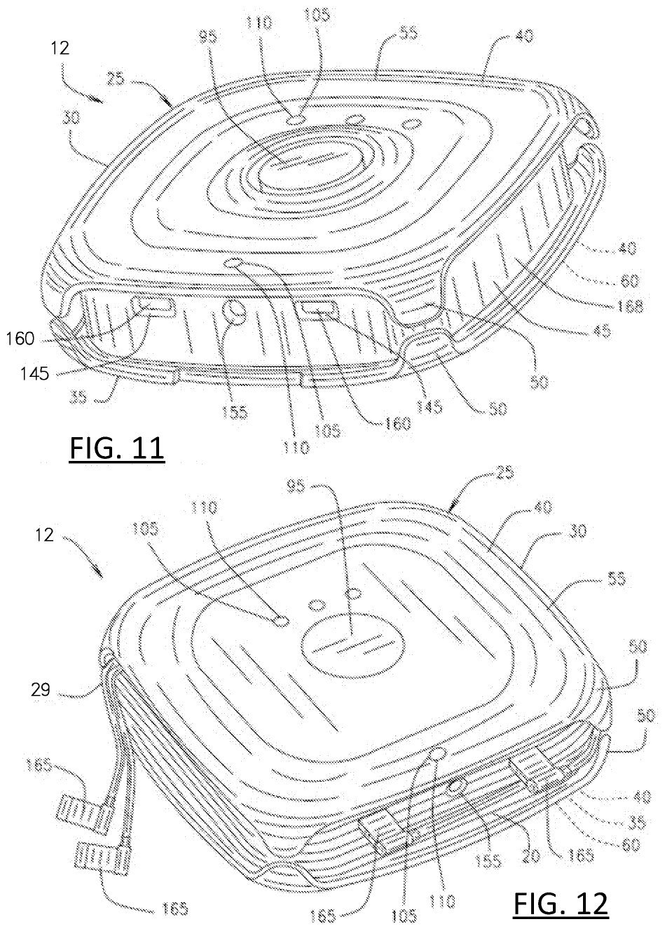

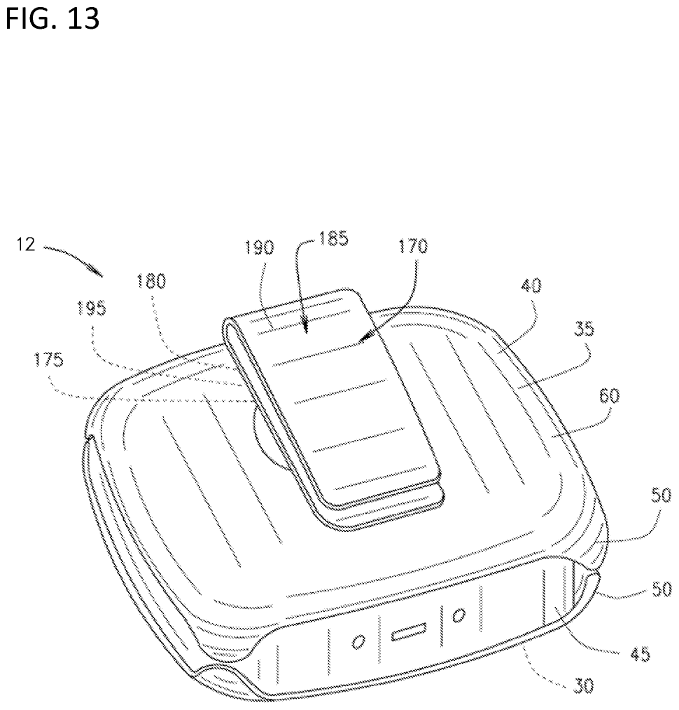

Referring to FIGS. 9-13 the electric controller 12 can be a portable controller, worn by a user in one or more embodiments. FIG. 9 illustrates a schematic diagram of an embodiment of the electric controller 12. In an embodiment, the controller 12 includes a microcontroller unit (MCU) 52, a low-dropout (LDO) regulator 54, a charging management circuit 56, an output control and temperature detecting circuit 58, an electrical power source 85, a selection mechanism 95, one or more indicators 100, a pinhead connector 155, and one or more flat connectors and temperature sensor 160. The MCU 52, LDO regulator 54, charging management circuit 56, output control and temperature detecting circuit 58, selection mechanism 95, indicators 100, pinhead connector 155, and flat connectors and temperature sensor 160 may be mounted on/within a printed circuit board 90 in an embodiment. The electrical power source 85 can include a chargeable electrical power supply, such as a battery or any USB power source, where electrical power to conductive heating element 6 is controlled by a power supply circuit. The power supply circuit can include the MCU 52 and the output control and temperature detecting circuit 58. An indicator 100 is provided, where the indication can indicate the status of the conductive heating element 6, on or off, or the status of the rechargeable electrical power source 85.

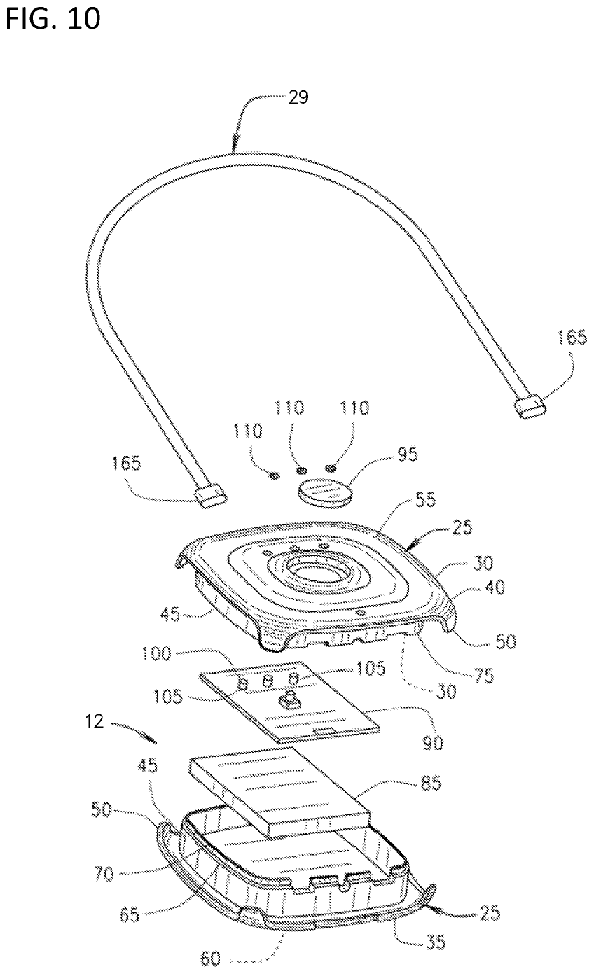

As illustrated in FIG. 10, the electric controller 12 includes a housing 25, which includes an upper shell 30 and a bottom shell 35 that engage one another to create the housing 25. In one embodiment, both the upper shell 30 and bottom shell 35 have an outer section 40 and an inner section 45, and when the upper shell 30 and bottom shell 35 engage one another, the inner sections 45 of both the upper shell 30 and bottom shell 35 are adjacent and abut one another. When assembled, the housing 25 therefore has two outer sections 40 and one inner section 45. In one embodiment, both the outer sections 40 and the inner section 45 are generally substantially rectangular members, although these components may be provided in any number of other shapes without significantly affecting the functionality of the overall system. In this embodiment, the outer sections 40 have a larger perimeter than the perimeter of the inner section 45. The housing 25 therefore preferably has a spool-like shape. The outer sections 40 may further have at least one securing section 50 that extends to secure at least one electrical connector 29. In one embodiment, the at least one securing section 50 includes the corners of the outer section 40 that are further curved or bent inwardly, towards the inner section 45. The at least one electrical connector 29 may therefore wrap around the inner section 45 of the housing 25 and be secured by the securing section 50 of the outer sections 40, as will be explained in greater detail hereinafter. However, in alternative embodiments, the housing 25 can be any shape and size as long as it is able to surround the components of the controller 12 and can secure the at least one electrical connector 29, which will be explained in greater detail hereinafter.

The upper shell 30 of the housing 25 is preferably the top face or outer face 55, while the bottom shell 35 is preferably the bottom face or the inner face 60, when the controller 12 is carried using a belt clip 170. In other words, when the controller 12 is worn by the human user, the bottom shell 35 is adjacent to the user and is generally not visible. The upper shell 30, on the other hand, is opposite from the bottom shell 35 and is visible to the user when the controller 12 is worn. In one embodiment, the bottom shell 35 further includes a bottom rim 65 and a lower rim 70, with a smaller perimeter than the perimeter of the bottom rim 65, and projects upwardly from the bottom rim 65 towards the upper shell 30 when the upper shell 30 and bottom shell 35 engage one another. Similarly, the upper shell 30 may also include an upper rim 75, where an upper lip 80 is set within the upper rim 75 to create a step within the upper shell 30, and the upper lip 80 further having a smaller perimeter than the perimeter of the upper rim 75. When the upper shell 30 engages with the bottom shell 35, the upper shell 30 is positioned over the bottom shell 35 where the bottom shell 35 is inserted into the upper shell 30, so that the bottom rim 65 of the bottom shell 35 and the upper rim 75 of the upper shell 30 abut and rest upon each other, while the lower rim 70 abuts and rests upon the upper lip 80. The upper shell 30 and bottom shell 35 therefore engage one another through a friction or press fit or in other embodiments may include a lip and at least one recess elements on the respective shells that are operable to engage one other in an interference fit to removably secure the two shells together. Other methods of engaging the upper shell 30 and bottom shell 35 are envisioned and foreseeable. The housing 25 is preferably made out of plastic, but can be any semi-rigid or rigid material instead.

The housing 25 of the controller 12 preferably serves to protect and contain the components of the controller 12. The components of the controller 12 include an electrical power source 85, which in one embodiment is a battery or battery pack, which preferably includes one or more lithium ion batteries. For example, a 7.4 V, 7000 mAh lithium ion battery capable of 8 hours of runtime. In another embodiment, the electrical power source 85 is a USB power source. The electrical power source 85 provides power to a printed circuit board 90, connected to a temperature selection mechanism 95, which may be an on/off button or switch. In a particularly preferred embodiment, the electrical power source 85 is a rechargeable battery to reduce waste, for example. The temperature selection mechanism 95 is preferably located and positioned on the outer face 55 of the housing 25, where the human user can push the temperature selection mechanism 95 to turn the power on or off, as seen in FIGS. 10 and 11. The temperature selection mechanism 95 can further be pushed to adjust the temperature setting of the controller 12 until the desired setting of low, medium, and high heat has been reached.

The controller 12 also includes at least one indicator 100 indicating at least one operation state of the controller 12 and/or electrical heating kinesiology tape 2 (e.g., the at least one operation variable may be when the controller 12 is on, the current temperature setting, or when the electrical power source 85 needs to be recharged). Thus, the controller 12 may include at least one battery status indicator, an on/off indicator, and a temperature setting indicator. In one embodiment, the at least one indicator 100 of the controller 12 may be visual indicators in the form of one or more light-emitting diode (LED) lights 105 that preferably will indicate whether the controller 12 has power, if it needs to be recharged, and its temperature setting. In one embodiment, the series of three LED lights 105 may turn on or change different colors, notifying the user of the selected pre-determined temperature setting. In the same or another embodiment, another LED light 105 may turn on or change different colors, notifying the user if the electrical power source 85 is fully charged, has a low charge, or if the electrical power source 85 has any charge remaining. The LED lights 105 may be covered and encased within at least one lens 110 to protect the LED lights 105 from being damaged, as can be seen in FIGS. 8-10. The lens 110 may be made out of plastic, but can be any material that is at least somewhat transparent so the user can see whether the LED light 105 is lit and/or the color of the LED light 105.

As illustrated in FIG. 10, the printed circuit board 90 and electrical power source 85 within the housing 25 are further connectable to at least one electrical connector 29 to provide electrical power to the electrical heating kinesiology tape 2, as explained in more detail herein.

As illustrated in FIGS. 11-13, the housing 25 further includes at least one female connector opening 145 capable of accepting at least one male plug 165. In one embodiment, the controller 12 preferably includes a female pinhead connector 155 that is capable of mating with a male pinhead plug connected to a cable and a cable plug, which is capable of mating with a wall outlet, therefore allowing the electrical power source 85 of the controller 12 to recharge. Therefore, when the electrical power source 85 of the controller 12 needs to be recharged, the human user may insert the male pinhead plug into the female pinhead connector 155 and plug the cable plug into the wall outlet to recharge the electrical power source 85. The controller 12 may further include at least one female flat connector 160 capable of mating with the male flat electric connection head 165 at both ends of each at least one electrical connector 29. The male flat electric connection head 165 at one end of the electrical connector 29 can be inserted into the female flat connector 160 in the controller 12, while the male flat electronic connection head 165 at the other end of the electronic connection 29 can be inserted into opposite free ends 18 and 20 of the conductive heating element 6 thereby allowing the controller 12 to be electrically coupled to the conductive heating element 6. In an embodiment, the opposite free ends 18 and 20 of the conductive heating element 6 are female connecting pins.

Once the male flat electronic connection head 165 has been inserted into the female flat connector 160 of the controller 12, the at least one electrical connector 29 may be wrapped around the inner section 45 of controller 12 to secure and hold the at least one electrical connector 29 out of the way, but still allow for easy access if the length of the at least one electronic connection 29 needs to be adjusted. In other words, the inner section 45 and outer section 40 of the controller engage and coordinate one another to define a recess 168 around the periphery of the controller where the at least one electrical connector 29 may be selectively wrapped around the inner section 45 of the housing 25. The outer sections 40 each further have at least one securing section 50 that extends to coordinate the securing of the electrical connection 29 around the recess or the inner section 45, and prevent the electrical connection 29 from unraveling when wrapped within the recess 168.

The inner face 60 of the controller 12 further includes a selectively engageable belt clip 170 so that a user may clip the controller 12 to a pair of pants or shorts, and use the electrical heating kinesiology tape 2 without being forced to remain in one place. In greater detail, the inner face 60 of the controller 12 includes at least one female snap button connector 175 located and positioned on the inner face 60 of the controller 12 that selectively engages at least one male snap button fastener 180 located and positioned on a holder 185 of the belt clip 170. The holder 185 is preferably an upside down U-shaped member where a user may slide his or her waistband in between the arms of the U-shaped member so that the holder 185 may selectively engage the waistband of a pair of pants. The holder 185 further has an inner side 190 and an outer side 195, where the inner side 190 is adjacent and abuts the user's undergarments if the user is using the belt clip 170 to facilitate carrying the controller 12. The outer side 195 of the holder 185 is adjacent to the inner face 60 of the controller 12 and includes the at least one male snap button fastener 180.

The at least one male snap button fastener 180 and at least one female snap button connector 175 are conventional type cooperatively engaging fasteners well known in the prior art. In operation, force is typically applied to the prongs of the male snap button fastener 180 so that the prongs are inwardly depressed when inserted into the female snap button connector 175. Once pressure is no longer applied to the prongs of the male snap button fastener 180, the prongs return to their normal position within the female snap button connector 175 thereby securing the male snap button fastener 180 within the female snap button connector 175. In order to disengage male snap button fastener 180 from the female snap button connector 175, the user applies force by pulling the male snap button fastener 180 away from the female snap button connector 175, thereby inwardly depressing the prongs and withdrawing the male snap button fastener 180 from the female snap button connector 175. The male snap button fastener 180 is therefore able to selectively engage with the female snap button connector 175, and the belt clip 170 therefore is selectively attachable and releasable from the controller 12.

In order to use the electrical heating kinesiology tape 2 when electrically coupled to the controller 12, the user may first check the at least one indicator 100 to determine whether the electrical power source 85 in the controller 12 has power or if the electrical power source 85 needs to be recharged or replaced, depending on the embodiment. In one embodiment where the at least one indicator 100 is a LED light 105, the LED light 105 can turn on, change colors, or otherwise signal that the electrical power source 85 has no or a low charge. If the electrical power source 85 needs to be recharged, the user may insert the cable plug into the wall socket and the male pinhead plug into the female pinhead connector 155 of the controller 12. The LED light 105 can further indicate that the electrical power source 85 is charging and when the electrical power source 85 is fully charged.

Once the electrical power source 85 has been sufficiently charged, the male pinhead plug may be removed from the female pinhead connector 155 of the controller 12, and the male flat electric connection head 165 of the at least one electrical connector 29 may be inserted into and mated to the at least one female flat connector 160 of the controller 12. The other male flat electric connection head 165 of the at least one electrical connector 29 may be connected to at least one of opposite free ends 18 and 20 of the conductive heating element 6 so that the male flat electric connection head 165 mates with the electrical heating kinesiology tape 2.

The electrical heating kinesiology tape 2 may be placed against the human user's skin so that the adhesive material 8 is adjacent and abuts the skin of the human user. Once the electrical heating kinesiology tape 2 has been positioned and attached at the desired area, the excess cable of the at least one electrical connector 29 may be wound around the inner section 45 of the controller 12 so that at least one electrical connector 29 does not get caught on other objects or become tangled. In an alternate embodiment, a split hard shell case is provided within which the electrical heating kinesiology tape 2 can be stored between uses. In a preferred version of this embodiment, the inside surfaces of the case (those against which the electrical heating kinesiology tape 2 may rest) are provided with multiple thin, raised fins that serve to minimize the surface contact between the shell and the electrical heating kinesiology tape 2.

If the user desires to move while using the electrical heating kinesiology tape 2, the user may have the belt clip 170 engage with the controller 12 by inserting the male snap button fastener 180 into the female snap button connector 175 on the inner face 60 of the controller 12. The user can then use the belt clip 170 to facilitate carrying the controller 12, and therefore is not required to carry the controller 12 by hand. The user can then press the temperature selection mechanism 95 to turn the controller 12 on and further press the temperature selection mechanism 95 to adjust the temperate setting to a pre-determined setting, if so desired. The controller 12 conducts the electrical power provided by the electrical power source 85 through the at least one electrical connector 29 to the electrical heating kinesiology tape 2 by engaging the conductive heating element 6 through the at least one electrical connector 29. The at least one electrical connector 29 is coupled to the output control and temperature detection circuit 58, which is further coupled to the electrical power source 85. The electrical connections extend from the controller 12 to the electrical heating kinesiology tape 2, where the electrical connectors 29 may be inserted into the controller 12 and connected to the conductive heating element 6. Thus, the electrical power source 85 is able to provide electrical power to the electrical heating kinesiology tape 2.

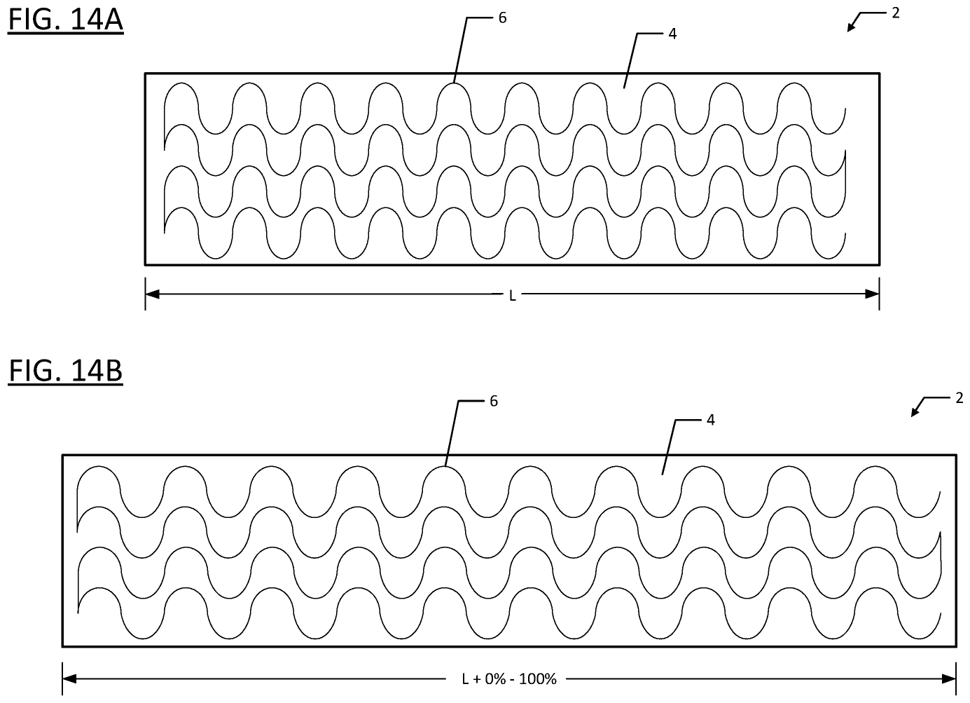

FIGS. 14A and 14B illustrate another exemplary embodiment of the stretchable heating tape 2 that is stretchable from 0%-100% of its non-stretched length “L”. In this exemplary embodiment, the conductive heating element 6 is not woven through the elastic backing material 4 but is instead affixed to one side of the elastic backing material 4. For example, the conductive heating element 6 may be affixed to the elastic backing material with one or more sewn threads or yarn or the like. Because no sections of the conductive heating element 6 are woven through the elastic backing material 4 in this exemplary embodiment, arcuate sections of the conductive heating element 6 can be affixed to the elastic backing material 4 and still accommodate stretching of the stretchable heating tape 2 without breaking.

The conductive heating element 6 illustrated in FIGS. 14A and 14B may alternatively be woven through the elastic backing material 4 as described above. In such an embodiment, the arcuate portions of the conductive heating element 6 would also be woven through the elastic backing material 4, in addition to the adjacent woven segments 14. As the stretchable heating tape 2 is stretched, its length elongates but its width narrows. The conductive heating element 6 is not directly affixed to any specific point of the elastic backing material 4, and is able to adjust its position therewithin. This allows the conductive heating element 6 to adjust its shape to conform to the stretched shape of the elastic backing material 6.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A stretchable heating tape comprising:
an elastic backing material having a first side and a second side;
a continuous heating element at least partially woven through the elastic backing material;
an adhesive material positioned on the second side of the elastic backing material; and
two or more heating element connection points positioned on the first side of the elastic backing material, the heating element connection points configured to enable electrically coupling an electric controller to the continuous heating element such that electric current from the electric controller is at least partially converted to heat energy when flowing through the continuous heating element.

2. A stretchable heating tape as set forth in claim 1, wherein the elastic backing material has a length and a width, wherein the continuous heating element is at least partially woven in a serpentine pattern along the length of the elastic backing material such that a plurality of woven segments of the continuous heating element each substantially span the width of the elastic backing material and such that a plurality of unwoven segments of the continuous heating element are unconstrained by the elastic backing material.

3. A stretchable heating tape as set forth in claim 1, wherein the elastic backing material has a length and a width, wherein the continuous heating element is at least partially woven in a serpentine pattern along the width of the elastic backing material such that a plurality of woven segments of the continuous heating element each at least span a portion of the length of the elastic backing material and such that a plurality of unwoven segments of the continuous heating element are unconstrained by the elastic backing material.

4. A stretchable heating tape as set forth in claim 1, wherein the elastic backing material has a length and a width, wherein the continuous heating element is at least partially woven in a serpentine pattern along the length of the elastic backing material such that a plurality of woven segments of the continuous heating element each at least span the length and the width of the elastic backing material and such that a plurality of unwoven segments of the continuous heating element are unconstrained by the elastic backing material.

5. A stretchable heating tape as set forth in claim 1, wherein the elastic backing material has a length and a width, wherein the continuous heating element is at least partially woven along the length of the elastic backing material such that a plurality of woven segments and a plurality of unwoven segments of the continuous heating element are formed along the length of the elastic backing material, wherein each of the plurality of woven segments is spaced apart from an adjacent woven segment.

6. A stretchable heating tape as set forth in claim 5, wherein each of the plurality of woven segments is spaced apart at least one inch from an adjacent woven segment.

7. A stretchable heating tape as set forth in claim 5, wherein each of the plurality of woven segments is spaced apart a distance less than one inch from an adjacent woven segment.

8. A stretchable heating tape as set forth in claim 5, wherein each of the plurality of woven segments is spaced apart a distance greater than one inch from an adjacent woven segment.

9. A stretchable heating tape as set forth in claim 1, wherein the elastic backing material is comprised of a single layer of anisotropically stretchable fabric knitted in a birds eye knitting stitch.

10. A stretchable heating tape as set forth in claim 1, wherein the elastic backing material and the adhesive material are configured to at least partially transfer the heat energy to a portion of a user's body when the adhesive material is positioned adjacent to the portion of the user's body.

11. A system, comprising:
a controller, comprising:
an electrical power source,
a selection mechanism, and
at least one indicator configured to indicate at least one operation state of the system;
a stretchable heating tape, comprising:
an elastic backing material having a first side and a second side,
a continuous heating element at least partially woven through the elastic backing material,
an adhesive material positioned on the second side of the elastic backing material, and
two or more heating element connection points positioned on the first side of the elastic backing material and electrically connected to the continuous heating element; and
an electrical connector that is selectively engageable with the controller and further selectively engageable with the two or more heating element connection points and operable to electrically couple the controller and the continuous heating element,
wherein the selection mechanism is operable to start and stop a flow of electric current from the electrical power source to the continuous heating element via the electrical connector and the two or more heating element connection points such that the electric current is at least partially converted to heat energy when flowing through the continuous heating element.

12. The system of claim 11, wherein the controller includes a housing having an inner section and an outer section, the inner section and the outer section coordinating to define a recess therebetween around the periphery of the controller within which the electrical connector is selectively wrappable around the inner section of the housing.

13. The system of claim 12, further including a belt clip that is selectively attachable and releasable from the housing.

14. The system of claim 11, wherein the electrical power source comprises at least one battery.

15. The system of claim 14, wherein the at least one battery is rechargeable.

16. The system of claim 11, wherein the elastic backing material has a length and a width, wherein the continuous heating element is at least partially woven in a serpentine pattern along the length of the elastic backing material such that a plurality of woven segments of the continuous heating element each substantially span the width of the elastic backing material and such that a plurality of unwoven segments of the continuous heating element are unconstrained by the elastic backing material.

17. The system of claim 11, wherein the elastic backing material has a length and a width, wherein the continuous heating element is at least partially woven in a serpentine pattern along the width of the elastic backing material such that a plurality of woven segments of the continuous heating element each at least span a portion of the length of the elastic backing material and such that a plurality of unwoven segments of the continuous heating element are unconstrained by the elastic backing material.

18. The system of claim 11, wherein the elastic backing material has a length and a width, wherein the continuous heating element is at least partially woven in a serpentine pattern along the length of the elastic backing material such that a plurality of woven segments of the continuous heating element each at least span the length and the width of the elastic backing material and such that a plurality of unwoven segments of the continuous heating element are unconstrained by the elastic backing material.

19. The system of claim 11, wherein the elastic backing material has a length and a width, wherein the continuous heating element is at least partially woven along the length of the elastic backing material such that a plurality of woven segments and a plurality of unwoven segments of the continuous heating element are formed along the length of the elastic backing material, wherein each of the plurality of woven segments is spaced apart from an adjacent woven segment.

20. The system of claim 11, wherein the elastic backing material is comprised of a single layer of anisotropically stretchable fabric knitted in a birds eye knitting stitch.
